(19) United States
(12) Patent Application Publication
(10) Pub. No.: US 2009/0177190 A1
(43) Pub. Date: Jul. 9, 2009

(54) Lowering skin melanin appearance with red light radiation and red light radiation kit therefor

(76) Inventor: Seung Yoon Lee, Kyeongki-do (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/291,669

(22) Filed: Nov. 12, 2008

Related U.S. Application Data
(60) Provisional application No. 61/003,508, filed on Nov. 16, 2007.

(51) Int. Cl. A61B 18/20

(52) U.S. Cl. 606/9

(57) ABSTRACT

A method of reducing appearance of melanin on the skin of a subject comprises exposing the skin to red narrow-band radiation at a wavelength(s) in a range of between 620 nm and 750 nm and having a band width of between 0 nm and 20 nm, in an effective dose to cause the appearance of the skin melanin to diminish and essentially not to cause photothermolysis of the skin. Alternatively, a method of reducing appearance of melanin on the skin of a subject comprises exposing the skin to non-coherent red narrow-band radiation at a wavelength(s) in a range of between 620 nm and 750 nm and having a band width of between 0.1 nm and 20 nm, in an effective dose to cause the appearance of the skin melanin. A portable kit for such a method comprises a radiation source generating red narrow-band radiation at a wavelength(s) in a range of between 620 nm and 750 nm, the narrow band radiation having a band width of between 0 nm and 20 nm and having a power density of between 10 mW/cm² and 120 mW/cm², and a manual instructing a user how to use the red narrow-band radiation for red narrow-band irradiation treatment to reduce appearance of melanin on the skin of a subject.

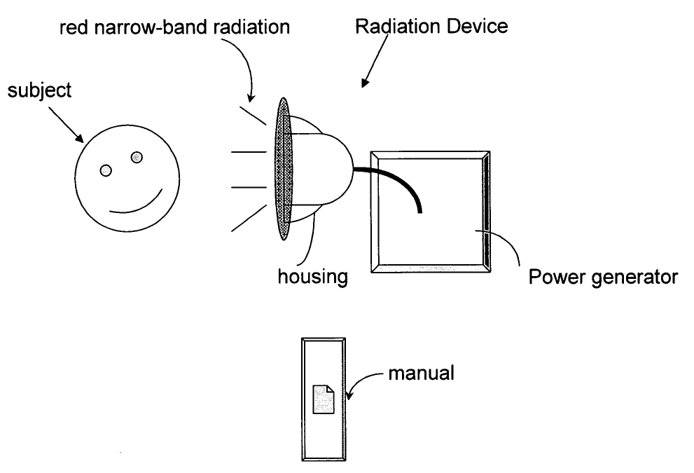

### RELATED APPLICATION

This application claims the benefit of U.S. Provisional Application No. 61/003,508, filed on Nov. 16, 2007. The entire teachings of the above application are incorporated herein by reference.

### BACKGROUND OF THE INVENTION

Uneven pigmentation generally is not appealing to most people. Also, certain people, in particular certain people having skin phototypes 3-5, desires to have overall relatively bright skin tones or skin complexion. Skin pigmentation and skin tones are generally affected by the contents and/or the appearance of melanin on the skin. Melanin is the pigment produced by melanocyte cells, and creates the color of skin, eyes and hair shades. The most typical cause of darkened areas of skin, brown spots or areas of discoloration is unprotected sun exposure.

A variety of topical treatment products for skin brightening are commercially available, such as topical lotions and gels containing melanin-inhibiting ingredients, along with a sunscreen. Examples of melanin-inhibiting ingredients include hydroquinone, kojic acid and arbutin. However, the effectiveness of such topical agents varies upon skin types and the degree of pigmentation problems. Also, certain melanin-inhibiting ingredients, such as mercury (II) chloride, hyroquinone and , kojic acid, are believed to have potential toxicity. Other treatments for brightening skin tones include chemical or physical peels to remove the outer layer of the skin. However, the results of such peels and laser treatments are not always consistent, and complications such as hypo- or hyperpigmentaion or infections can occur. In particular, it has been reported that laser treatments are more likely to result in problems for those with darker skin phototypes. In addition, laser treatments generally require intensive post-treatment care, and can lead to considerable complications including long-lasting erythema, pain, infection, bleeding, hyper- or hypopigmentaion and sometimes scarring.

Thus, there is a need for developing methods for lowering appearance of melanin on the skin, in particular relatively effective, safe, well-tolerated, and painless treatment methods.

### SUMMARY OF THE INVENTION

Applicant has now discovered that red light irradiation at a wavelength(s) in a range of between 620 nm and 750 nm at a relatively low power, such as between 10 mW/cm² and 120 mW/cm², can effectively reduce the appearance of melanin on the skin of a subject. Based on this discovery, a method of lowering appearance of melanin on the skin with the red light irradiation and a kit for such a method of lowering the appearance of melanin on the skin with the red light irradiation are disclosed herein.

In one embodiment, the present invention is directed to a method of reducing appearance of melanin on the skin of a subject. The method comprises exposing the skin to red narrow-band radiation at a wavelength(s) in a range of between 620 nm and 750 nm and having a band width of between 0 nm and 20 nm, in an effective dose to cause the appearance of the skin melanin to diminish and essentially not to cause photothermolysis of the skin.

In another embodiment, the present invention is directed to a method of reducing appearance of melanin on the skin of a subject. The method comprises exposing the skin to red non-coherent narrow-band radiation at a wavelength(s) in a range of between 620 nm and 750 nm and having a band width of between 0.1 nm and 20 nm, in an effective dose to cause the appearance of the skin melanin to diminish.

In yet another embodiment, the present invention is directed to a kit that comprises a radiation source generating red narrow-band radiation at a wavelength(s) in a range of between 620 nm and 750 nm, the narrow band radiation having a band width of between 0 nm and 20 nm and having a radiation power of between 10 mW/cm² and 120 mW/cm². The kit further comprises a manual instructing a user how to use the red narrow-band radiation for red narrow-band irradiation treatment to reduce appearance of melanin on the skin of a subject.

The present invention also includes use of red narrow-band radiation at a wavelength(s) a range of between 620 nm and 750 nm and having a band width of between 0 nm and 20 nm, in an effective dose to cause the appearance of the skin melanin to diminish and not to cause photothermolysis of the skin.

The present invention has several advantages. For example, the reduction of the appearance of melanin on the skin of a subject can be effectively achieved in a non-invasive way. In particular, a relatively non-thermal, red light-emitting diode (LED) radiation source can be employed in the invention, which provides an additional advantage that the reduction of the appearance of melanin of the skin can be effectively achieved in a non-thermal way. In addition, since LED radiation is relatively safe, patients can perform the treatment at their home. Also, microdermabrasion or exfoliating of the skin, or chemical peel can be employed prior to the red narrow-band radiation treatment. The beneficial effect associated with the peel can enhance the effect of lowering the skin melanin appearance, because the peel can remove excessive stratum corneum, or exfoliate a skin surface, and generate a relatively even skin surface. As a consequence, light scattering by excessive stratum corneum can be reduced. The reduction in light scattering can in turn allow as many photons as available at a given energy density of the red narrow-band radiation can penetrate into the skin, and thus, reduction of the skin melanin appearance can be obtained more efficiently. Moreover, a thin layer of transparent water-based gel on the skin prior to the red narrow-band radiation treatment can be employed. The transparent water gel layer can make a surface through which relatively large amounts of photons of the red narrow-band radiation can penetrate, and thus, reduction of the skin melanin appearance can be obtained effectively with relatively low power density and/or less duration time. In addition, the thin layer of transparent water-based gel can prevent potential dehydration of the skin surface subject to the irradiation treatment and can keep the skin hydrated during the treatment. Furthermore, application of skin-brightening cosmetics before or after the red narrow-band radiation treatment can enhance the effect of lowering the skin melanin appearance.

### BRIEF DESCRIPTION OF THE DRAWING

The FIGURE is a schematic drawing showing a kit of the invention that includes a radiation device generating the red narrow-band radiation employed in the invention, and a manual instructing a user how to use the red narrow-band radiation for red narrow-band irradiation treatment to reduce the appearance of melanin on the skin of a subject.

### DETAILED DESCRIPTION OF THE INVENTION

The invention employs red narrow-band radiation at a wavelength(s) in a range of between 620 nm and 750 nm and having a band width of between 0 nm and 20 m in an effective dose to cause the appearance of melanin on the skin of a subject to diminish.

As used herein, the “appearance” of melanin on the skin of a subject is a melanin level obtained based upon the amount of beam reflected back from the skin relative to the amount of reference beam irradiated onto the skin. Such amount can be measured by any suitable method known in the art, such as a Mexameter™, which is an objective skin color-measuring device, and has been reported to provide a reproducible and sensitive means of quantifying small skin color differences. For example, a Mexameter™ measures the amount of beam reflected back from the skin relative to the amount of reference beam irradiated onto the skin, and calculates a melanin level in an indicative index (e.g., when given two calculated indexes are 100 and 150; index 150 means darker skin tone than index 100). With the lowering of the appearance of melanin on the skin of a subject by a method of the invention, the subject perceives that the skin color gets less dark (or brightened) after the red narrow-band radiation treatment than before the treatment.

“Narrow band” radiation, as used herein, means radiation at a wavelength or wavelengths having a band width between 0 nm and 20 nm. It is noted that the term “between 0 nm and 20 nm” includes “0 nm” and “20 nm.” For example, narrow band radiation at a wavelength(s) having a band width of 20 nm means that the radiation at the wavelength(s) has, for example, a deviation of ±10 nm. Similarly, narrow band radiation at a wavelength(s) having a band width of 0 nm means that the radiation at the wavelength(s) has a deviation of ±0 nm.

As used herein a “subject” is a mammal, preferably a human. Subject and patient are used interchangeably.

As used herein, the “effective dose” is a dose sufficient to cause statistically significant reduction, or reduction of the appearance of melanin noticeable enough for the subjects to perceive themselves, in the appearance of melanin on the skin after each dose or after a plurality of consecutive such doses. For example, the “effective dose” is a dose sufficient to cause statistically significant reduction of 1%, 5%, 10%, 15%, 20%, etc. in the melanin levels obtained by a Mexameter™ in the appearance of melanin on the skin after each dose or after a plurality of consecutive such doses. When a plurality of consecutive doses are employed, the doses are typically repeated at intervals of from 0.5 day (twice per day) to 10 days. Specifically, the doses are repeated at intervals of from one day to 4 days, such as 2 days or 3 days. Alternatively, each irradiant dose is up to 7 days apart, such as 1 day apart, 2 days apart, 3 days apart or 4 days apart. More specifically, each irradiant dose is 1 day apart, 2 days apart or 3 days apart. The intervals can be the same length or different lengths. In one specific embodiment, the intervals are the same length.

In one embodiment, the effective dose essentially does not cause photothermolysis of the skin. Selective photothermolysis is a photothermolytic reaction by which a target chromophore is selectively damaged or destroyed by light, resulting in destruction of the target chromophore or necrosis of the cells that contain the target chromophore. Photothermolysis generally occurs when the following three fundamental conditions are met:
Wavelength: specific wavelength that can be absorbed by the target molecule;
Pulse duration: pulse duration of the pulsed light from a laser that is shorter than the thermal relaxation time of the target (TRT: TRT is the time taken for the target to dissipate about 63% of the incident thermal energy); and
Fluence (energy density, J/cm²): a sufficient fluence (energy density; the amount of energy per unit area) to create the thermal damage enough to destroy the target.
   In one example, meeting the above-mentioned three conditions, when the pulsed light from a laser is absorbed by a given target within time duration shorter than the TRT of the target (thus, the pulse duration of the pulsed light should be shorter than the TRT of the target), the target cannot dissipate the heat energy to the adjacent structures before the sufficient amount of energy to destroy it accumulates in it, and therefore, is destroyed by the thermal damage. For example, the TRT of the melanosome (melanin pigments within the malanocytes) is 0.5-1 microsecond (10⁻⁶ second).

Typically, photothermolysis can occur with a laser light source that can produce short pulses. For melanin pigments, the TRT is so short that only specific lasers that are equipped with a special device named Q-switch (quality switch), which can make very high energy, ultra-short pulses in the range of nanoseconds, can induce photothermolysis of melanin pigments. Q-switching, which is generally also known as giant pulse formation, is a technique by which a laser can be made to produce a pulsed output beam having very short pulses. The technique allows the production of light pulses with extremely high peak power, much higher than would be produced by the same laser if it were operating in a continuous wave (constant output) mode. Q-switching is generally achieved by putting a variable attenuator inside the laser's optical resonator. Generally, a high Q factor corresponds to low resonator losses per roundtrip, and vice versa. The variable attenuator is commonly called a “Q-switch”, when used for this purpose. Initially the laser medium is pumped while the Q-switch is set to prevent feedback of light into the gain medium (producing an optical resonator with low Q). This produces a population inversion, but laser operation cannot yet occur since there is no feedback from the resonator. Therefore, the amount of energy stored in the gain medium increases as the medium is pumped. At a certain time the stored energy will reach some maximum level; the medium is said to be gain saturated, the Q-switch device is quickly changed from low to high Q, allowing feedback and the process of optical amplification by stimulated emission to begin. Because of the large amount of energy already stored in the gain medium, the intensity of light in the laser resonator builds up very quickly; this also causes the energy stored in the medium to be depleted almost as quickly. The net result is a short pulse of light output from the laser, known as a giant pulse, which may have a very high peak intensity.

In general, a fluence that causes photothermolysis varies depending upon the types of the target, TRT of the target, depth of the target, type of lasers, skin phototypes of the subjects, and many other things, because, at least in part, power sufficient for photothemmolysis varies depending upon the target, laser type, depth of the target, skin phototypes, etc. In a particular example, when the TRT of a given melanin particle is 1 microsecond (10⁻⁶), if the sufficient energy density is 2 J/cm², the required power density is 2 J/cm² divided by 10⁻⁶ seconds, which yields 2,000,000 watts/cm². This power density generally cannot be produced with an LED light source or with a laser source unequipped with a Q-switch. Thus, with an LED light source or a low level laser unequipped with a Q-switch, selective photothermolysis of melanin pigments generally does not occur.

In one embodiment, the red narrow-band radiation employed in the invention does not meet at least one of the above-mentioned three requirements for photothermolysis.

In another embodiment, the red narrow-band radiation employed in the invention is generated from an LED light source or a low level laser without Q switching (e.g., a low level laser unequipped with a Q switch; or a low level laser equipped with a Q switch, but without using a Q switch).

Typically, in the disclosed methods, the skin of the subject is exposed to a plurality of exposures. The exposures can be repeated for any time period, as long as the subject does not experience any side effect, such as photosensitity. The plurality of exposures are collectively referred to as a “treatment period.” The treatment period can be between one week and 12 weeks, or between two weeks and 8 weeks, such as two, three, four, five or six weeks. Alternatively, the treatment period can be longer than 12 weeks.

In one embodiment, the skin is exposed to the red narrow-band radiation one, two, three, four, five, six or seven times per week during the treatment period. In another embodiment, the skin is exposed to the red narrow-band radiation four, five, six or seven times per week during the treatment period. In yet another embodiment, the skin exposure to narrow band radiation during the treatment period is only limited to the red narrow-band radiation at a wavelength(s) in a range of between 620 nm and 750 nm and having a band width of between 0 nm and 20 nm.

In one specific embodiment, the exposures are performed other than three times a week for three week.

In yet another specific embodiment, exposure of the skin to narrow band radiation at a wavelength(s) outside of the range between 620 nm and 750 nm, up to a week prior to the beginning of treatment period and after the end of treatment period, is with an energy density less than 1 J/cm².

In yet another specific embodiment, exposure of the skin to narrow band radiation at a wavelength(s) greater than 750 nm, up to a week prior to the beginning of treatment period and after the end of treatment period, is in an amount less than 1 J/cm².

In yet another specific embodiment, during the treatment period, e.g., between skin exposures to the red narrow-band radiation, exposure of the skin to narrow band radiation at a wavelength(s) outside of the range between 620 nm and 750 nm is with an energy density of less than 1 J/cm².

In yet another specific embodiment, during the treatment period, e.g., between skin exposures to the red narrow-band radiation, exposure of the skin to narrow band radiation at a wavelength(s) greater than 750 nm is in an amount less than 1 J/cm².

In yet another specific embodiment, the skin exposed to the red narrow-band radiation is skin from other than the face of the subject, such as the neck, the shoulder, the arms, etc.

In yet another specific embodiment, the skin exposed to the red narrow-band radiation is essentially free of wrinkles.

Generally, an effective dose of the red narrow-band radiation depends, in each case, upon several factors, e.g., the skin types (especially skin phototypes), age, gender and condition of the subject to be treated, among others. In one embodiment, the exposure time and/or power density of the red narrow-band radiation is adjusted according to the subject's skin conditions, particularly the skin phototype of the subject. For example, individual subjects' skin phototypes, conditions, and sensitivity or reactions to light can be different from each other. Skin phototypes of subjects to be treated can be classified by the Fitzpatrick skin phototype scale shown below:

TABLE 1
Fitzpatrick's Classification of Sun-Reactive Skin Types
Fitzpatrick's classification of sun-reactive skin types

| Skin | | |
|---|---|---|
| Phototype* | Unexposed skin color | Sun responses history |
| I | White | Always burn, never tan |
| II | White | Usually burn, tan with difficulty |
| III | White | Sometimes mild burn, tan average |
| IV | Moderate brown | Rarely burn, tan with ease |
| V | Dark brown** | Very rarely burn, tan very easily |
| VI | Black | No burn, tan very easily |

*Based on the first 30-60 minutes of sun exposure of untanned skin after the winter season
**Asian Indian, Oriental, Hispanic, or light African descent, for example

The Fitzpatrick' classification of skin phototypes of a given subject can influence the degree of absorption of the photons by the skin cells, and therefore, the degree of effect of lowering skin melanin appearance. In a specific embodiment, exposure time and/or radiation power of the red narrow-band radiation is increased as the skin phototype of the subject varies from skin phototype I to skin phototype VI of the Fitzpatrick's classification of skin phototypes.

In another embodiment, the red narrow-band radiation treatment is employed in combination with one or more peeling means, such as superficial peeling means, known in the art. In a specific embodiment, at least one of microdermabrasion, exfoliating and chemical peel of the skin of the subject is performed prior to the skin exposure to the red narrow-band radiation. Suitable peeling means include physical means for microdermabrasion, physical means for exfoliating of the skin surface, and chemical peeling means. Generally, superficial peels remove part or all of the epidermis, which is then followed by the natural wound healing process. For example, very light superficial peels generally involve to the level of the stratum spinosum, and light superficial peels generally involve the entire epidermis. Generally, medium depth peels involve the entire epidermis plus the papillary dermis to the level of the upper reticular dermis. The microdermabrasion, exfoliating or chemical peel of the skin of the subject can be performed by any suitable method known in the art. Specific examples include diamond peels, crystal peels, skin scrubbers with ultrasonic waves, an exfoliating means (e.g., washable cream including fruit seeds or buffing beads), physical means of superficial skin resurfacing, and chemical peels.

Microdermabrasion (often referred to as microderm) is a cosmetic procedure in which the stratum corneum (dead outermost surface of the skin) is partially or completely removed by light abrasion, such as mechanical abrasion using jets of zinc oxide or aluminum oxide crystals, fine organic particles, or a roughened surface. Superficial skin resurfacing includes laser superficial resurfacing.

Chemical peels can be done with a composition comprising a pharmaceutically acceptable peeling agent(s), such as salicylic acid or glycolic acid. In a specific embodiment, the composition comprises trichloroacetic acid, resorcinol, salicylic acid, lactic acid, an alpha-hydroxy acid, a beta-hydroxy acid and a seaweed extract including an enzymatic exfoliating agent. Examples of alpha-hydroxy acids include lactic acid, glycolic acid, malic acid, citric acid and tartaric acid. Examples of beta-hydroxy acids include salicylic acid, benzoic acid and buteric acid. One example of chemical peel compositions comprises 10-20% trichloroacetic acid, alpha-hydroxy acid, beta-hydroxy acid and tretinoin. Another example of chemical peel compositions comprises 20-30% trichloroacetic acid, Jessner's solution, a modified Jessner's solution and glycolic acid. Jessner's solutions includes resorcinol, salicylic acid, 85% lactic acid and 95% ethanol. Modified Jessner's solution including these ingredients with other concentrations can also be employed. Additional examples of chemical peel compositions include fruit extracts containing alpha-hydroxy acid, food extracts containing alpha-hydroxy acids, and plant extracts containing alpha-hydroxy acids or beta-hydroxy acids.

In one specific embodiment, a chemical peel is employed in the invention. Specific examples of suitable chemical peeling agents are as described in the previous paragraph.

In yet another embodiment, a layer of a gel, cream or lotion is applied on the skin prior to the skin exposure to the red narrow-band radiation. Thus, in this embodiment, the skin is exposed to the red-narrow band radiation through the gel, cream or lotion layer. Specifically, the gel, cream or lotion has at least 70% transparency at the red narrow-band radiation. More specifically, the gel, cream or lotion has at least 90% transparency at the red narrow-band radiation. In another specific embodiment, a layer of a transparent gel having, for example, at least 70% transparency, particularly at least 90% transparency, at the red narrow-band radiation is applied to the skin prior to the skin exposure to the red narrow-band radiation. In a further specific embodiment, the transparent gel is water-based. More specifically, the water-based transparent gel comprises hyaluronic acid. Even more specifically, the water-based transparent gel consists essentially of water and hyaluronic acid.

In yet another embodiment, the red narrow-band radiation treatment is performed in combination with one or more skin-brightening cosmetic products, prior to, and/or after, the skin exposure to the red narrow band radiation. Any suitable brightening cosmetic product that includes one or more skin-brightening agents known in the art can be employed in the invention. Specific examples of suitable skin-brightening agents include an alpha-hydroxy acid or a beta-hydroxy acid. Other specific examples of suitable skin-brightening agents include hydroquinone, kojic acid, azelaic acid, licorice P-T, arbutin, melawhite, ascorbic acid, vitamin C, magnesium-L-ascorbyl-2-phosphate and corticosteroid. Specific examples of the alpha-hydroxy acids and beta-hydroxy acids are as described above. Generally, the concentrations of alpha-hydroxy acids and beta-hydroxy acids in the cosmetic compositions are relatively low compared to those of chemical peel compositions. In some more specific embodiments, the cosmetic products do not comprise retinoic acid or retinoic acid derivatives. Not being bound to a particular theory, it is believed in the art that retinoic acid or retinoic acid derivatives can induce photosensitivity, causing skin irritation when exposed to light. However, the use of cosmetic products including retinoic acid or retinoic acid derivatives after the red narrow-band radiation treatment is not essentially limited.

In the invention, the red narrow-band radiation is at a wavelength(s) in a range of between 620 nm and 750 nm. In any one of the embodiments described above, alternatively, the red narrow-band radiation is at a wavelength(s) in a range of between 625 nm and 700 nm. Alternatively, the red narrow-band radiation is at a wavelength(s) in a range of between 625 nm and 680 nm in any one of the embodiments described above. Alternatively, the red narrow-band radiation is at a wavelength(s) in a range of between 625 nm and 650 nm in any one of the embodiments described above. Alternatively, the red narrow-band radiation is at a wavelength(s) in a range of between 627 nm and 639 nm in any one of the embodiments described above. More specifically, the red narrow-band radiation is at 633 nm in any one of the embodiments described above.

Typically, the red narrow-band radiation has a band width of between 0 nm and 20 nm. Specifically, in any one of the embodiments described in the previous paragraph, the band width of the red narrow band radiation is between 0 nm and 15 nm, such as 0 nm, 6 nm, 10 nm, 12 nm or 15 nm. More specifically, in any one of the embodiments described in the previous paragraph, the band width of the red narrow band radiation is between 0 nm and 12 nm. Alternatively, in any one of the embodiments described in the previous paragraph, the band width of the red narrow band radiation is between 0.1 nm and 12 nm. Alternatively, in any one of the embodiments described in the previous paragraph, the band width of the red narrow band radiation is between 0.1 nm and 1 nm.

In any one of the embodiments described in the two previous paragraphs, specifically, the red narrow-band radiation has power density in a range of between 10 mW/cm² and 120 mW/cm². More specifically, the power density is in a range of between 10 mW/cm² and 75 mW/cm² in any one of the embodiments described in the two previous paragraphs. Even more specifically, the power density is in a range of between 10 mW/cm² and 50 mW/cm² in any one of the embodiments described in the two previous paragraphs.

In any one of the embodiments described above, including the embodiments described in the three previous paragraphs, specifically, the red narrow-band radiation has energy density in a range of between 10 J/cm² and 200 J/cm². More specifically, the energy density is in a range of between 10 J/cm² and 120 J/cm². Alternatively, the energy density is in a range of between 10 J/cm² and 100 J/cm². Alternatively, the energy density is in a range of between 10 J/cm² and 70 J/cm². Even more specifically, the energy density is in a range of between 10 J/cm² and 75 J/cm², or between 35 J/cm² and 75 J/cm².

The skin exposure to the red narrow-band radiation per each dose can last for any suitable time period as long as it can cause the appearance of melanin on the skin to diminish after each dose or after a plurality of such doses, and essentially not to cause photothermolysis of the skin. Specifically, in any one of the embodiments described above, the skin exposure to the red narrow-band radiation per each dose lasts for less than 20 minutes. More specifically, in any one of the embodiments described above, the duration of the skin exposure to the red narrow-band radiation per each dose is between 5 minutes and 20 minutes, or between 5 minutes and 15 minutes, such as 10 minutes. Alternatively, in any one of the embodiments described above, the skin exposure to the red narrow-band radiation per each dose can last for more than 20 minutes, for example, between 20 minutes and 60 minutes or between 20 minutes and 40 minutes. Particularly, when the power density is in a range of between 10 mW/cm² and 75 mW/cm², or between 10 mW/cm² and 50 mW/cm², the duration of the skin exposure to the red narrow-band radiation per each dose can last for more than 20 minutes, for example, can be in a range of between 10 minutes and 60 minutes or between 10 minutes and 30 minutes.

For the red narrow band radiation employed in the invention, any suitable radiation source can be employed, including relatively low-power laser and light-emitting diodes (LEDs) known in the art. Specifically, the red narrow-band radiation employed in the invention is non-coherent radiation. More specifically, the red narrow-band radiation employed in the invention is generated by a red LED device.

Generally, prior to performing the red narrow-band radiation treatment of the invention, it is generally checked whether or not the subject to be treated has any contraindication (absolute and relative), such as photosensitive condition, especially in regards to any possibility of photosensitivity. Examples of absolute contraindication include: recent history (within one weak) of systemic or topical photodynamic therapy involving any photosensitizer that has the peak absorption within the range of red light waveband (e.g. 5-aminolevulinic acid, methyl-5-aminolevulinic acid) and the use of such photosensitizer for any other purposes. Examples of relative contraindication include: any photosensitive condition, such as disease (e.g. systemic lupus erythematosus, certain types of porphyria (erythropoietic porphyria, erythropoietic protoporphyria, porphyria cutanea tarda, variegate porphyria, hereditary coproporphyria, hepatoerythropoietic porphyria), polymorphous light eruption, hydroa vacciniforme, and other conditions that can cause photosensitivity), drugs (e.g. tetracycline, fluoroquinolones, ibuprofen, amiodarone, phenothiazine, furosemide, hydrochlorothiazide, retinoic acid, isotretinoin, etc.), and certain neurologic diseases that can be aggravated by light stimuli (certain types of epilepsy or other seizure disorders). For example, it is generally checked whether or not the subject to be treated has photosensitive condition, especially in regards to any possibility of photosensitivity, such as disease (e.g. systemic lupus erythematosus, certain types of porphyria (erythropoietic porphyria, erythropoietic protoporphyria, porphyria cutanea tarda, variegate porphyria, hereditary coproporphyria, hepatoerythropoietic porphyria), polymorphous light eruption, hydroa vacciniforme, and other conditions that can cause photosensitivity), drugs (e.g. tetracycline, fluoroquinolones, ibuprofen, amiodarone, phenothiazine, furosemide, hydrochlorothiazide, retinoic acid, isotretinoin, etc.), recent history of photodynamic therapy using 5-aminolevulinic acid or methyl-5-aminolevulinic acid or photofrin or other photosensitizers, certain neurologic diseases that can be aggravated by light stimuli (certain types of epilepsy or other seizure disorders). If the subject has one or more of these conditions or other photosensitive conditions, it is recommended for the subject to consult her/his doctor regarding whether or not, and/or when, she/he can take the red narrow-band radiation treatment of the invention.

The invention also includes a kit comprising a radiation source generating the red narrow-band radiation employed in the red narrow-band radiation methods described above. Specifically, the red narrow-band radiation has power density in a range of between 10 mW/cm² and 120 mW/cm². More specifically, the power density is between 10 mW/cm² and 75 mW/cm². Even more specifically, the power density is between 10 mW/cm² and 50 mW/cm². The kit further comprises a manual instructing a user how to use the red narrow-band radiation for the red narrow-band irradiation treatment to reduce appearance of melanin on the skin of a subject. Features, including specific features, of the red narrow-band irradiation treatment using the kit are as described above for the methods of the invention.

The FIGURE shows one embodiment of a kit of the invention, comprising a radiation device, such as an LED device, and a manual instructing a user how to use the red narrow-band radiation for the red narrow-band irradiation treatment to reduce appearance of melanin on the skin of a subject. The housing of the radiation device of the FIGURE can include any suitable radiation source, such as one or more red LEDs.

In one embodiment of a kit of the invention, the radiation source is an LED light source or a low level laser without Q switching (e.g., a low level laser unequipped with a Q switch; or a low level laser equipped with a Q switch, but without using a Q switch).

In a specific embodiment, the radiation source is a non-coherent radiation source, such as an LED device that includes one or more LEDs, preferably red LEDs.

In another embodiment, the manual included in a kit of the invention further comprises instructions about distance between the skin of the subject and the radiation source during the red narrow-band radiation treatment, duration time per single treatment of the red narrow-band radiation and frequency of the red narrow-band radiation treatment, and warning about contraindication (absolute and relative) of the red narrow-band radiation treatment. Specific examples of absolute and relative contraindication are as described above. In a further specific embodiment, the warning about the contraindication of the red narrow-band radiation treatment recommends users or subjects that they should seek professional advice as to whether the subject(s) to be treated has any photosensitive condition prior to using the kit for the red narrow-band radiation treatment, if they do not have prior knowledge about this. In an even further specific embodiment, the warning includes a statement that users or subjects should seek special consult's advice in regards to any possibility of photosensitivity, such as disease (e.g. systemic lupus erythematosus, certain types of porphyria (erythropoietic porphyria, erythropoietic protoporphyria, porphyria cutanea tarda, variegate porphyria, hereditary coproporphyria, hepatoerythropoietic porphyria), polymorphous light eruption, hydroa vacciniforme, and other conditions that can cause photosensitivity), drugs (e.g. tetracycline, fluoroquinolones, ibuprofen, amiodarone, phenothiazine, furosemide, hydrochlorothiazide, retinoic acid, isotretinoin, etc.), recent history of photodynamic therapy using 5-aminolevulinic acid or methyl-5-aminolevulinic acid or photofrin or other photosensitizers, certain neurologic diseases that can be aggravated by light stimuli (certain types of epilepsy or other seizure disorders). The subjects who have these conditions or other photosensitive conditions, or the subjects who learn that they have these conditions from their medical consult should seek special medical advice about using the red narrow-band radiation.

In another further specific embodiment, the manual further includes a chart of skin phototypes classification (e.g., the Fitzpatrick' classification of skin phototypes), and instructions about how to adjust exposure time and/or power density of the red narrow-band radiation according to the skin phototypes.

In one specific embodiment, the kit further comprises one or more peeling means, and/or a gel, cream, or lotion having at least 70% transparency at the red narrow-band radiation. Specific examples of suitable peeling means and transparent gels, creams or lotions are as described above for the methods of the invention. Specifically, the gel, cream or lotion included in the kit has at least 90% transparency at the red narrow-band radiation. More specifically, a transparent gel having at least 70% transparency, particularly at least 90% transparency, at the red narrow-band radiation is employed. Even more specifically, a water-based transparent gel having at least 70% transparency, particularly at least 90% transparency, at the red narrow-band radiation is employed.

In another specific embodiment, the kit further comprises a skin-brightening cosmetic product that includes one or more skin-brightening agents. Specific examples of skin-brightening agents are as described above for the methods of the invention. In yet another specific embodiment, the kit further comprises one or more peeling means, and/or a gel, cream, or lotion having at least 70% transparency at the red narrow-band radiation, and further comprises one or more skin-brightening cosmetic products.

In yet another specific embodiment, the kit further comprises a pair of goggles that are specifically designed to protect the retinae of the eyes of the subject to be treated with the red narrow-band radiation from direct illumination at the wavelength(s) of the red narrow-band radiation. Specifically, the goggles have color and/or optical density to essentially block light at the wavelength(s) of the red narrow-band radiation.

In yet another specific embodiment, the kit further comprises a device that measures the melanin level of the skin. Any suitable device that can measure a melanin level of the skin, based upon the amount of beam reflected back from the skin relative to the amount of reference beam irradiated onto the skin, can be employed in the invention, such as a Mexameter™.

In a preferred embodiment, the kit is portable. More preferably, the kit is a home-therapy kit so that a user is the subject to be treated with the red narrow-band radiation. In a specific embodiment of the home-therapy kit, the radiation source is an LED device that includes one or more LEDs, preferably red LEDs. In a more specific embodiment of the home-therapy kit, the LED device generates the red narrow-band radiation having power density in a range of between 10 mW/cm² and 75 mW/cm², or between 10 mW/cm² and 50 mW/cm².

The invention is illustrated by the following examples which are not intended to be limiting in any way.

### EXEMPLIFICATION

### Example 1

### Skin Melanin Level Lowering Effects of Red Light LED Irradiation: Once a Week

### Light Source

The phototherapy system used as the light source for this study consisted of a base and interchangeable heads emitting quasimonochromatic light of each different preset wavelength from adjustable planar arrays of LEDs. Red and Blue wavebands were employed in this study. The head emitting red light (Omnilux Revive™, Photo Therapeutics Ltd., Fazeley, UK) comprised four articulated panels containing 420 LEDs each, so that they could be adjusted to fit the contour of the patient's face optimally. The blue light head (Omnilux Blue™, Photo Therapeutics Ltd., Fazeley, UK) consisted of five panels containing 260 LEDs each arranged in the same way. The treatment heads delivered symmetrical peak wavelengths; 415±5 nm for the blue light and 633±6 nm for the red light. The irradiance was 40 mW/cm² for the blue light and 80 mW/cm² for the red light at a distance of 1 to 10 centimeters from the light source. The radiant fluences, or doses, during a single treatment for twenty minutes were 48 J/cm² and 96 J/cm² for the blue and red treatment heads, respectively.

### Study Design

Twenty seven patients of both sexes with mild to moderately severe facial acne were recruited for this study. The patients visited our clinic with all make-up removed and rested in a stable environment for about fifteen minutes. A dermatologist carried out objective instrumental measurements of the moisture level, the sebum level, and the melanin level of the patient's facial skin. After the measurements, each patient washed his or her face with a gentle soap and was treated for twenty minutes in the supine position. The irradiating head was positioned about 3-5 cm above from the patient's nose, and the articulated panels comprising the head were adjusted to match the contour of the patient's face. Goggles were worn during the treatment to protect the retinae from direct illumination. When the treatment was over, the instrumental measurements were done in the same way as before treatment, which signaled the end of one treatment session. In this manner, the therapy was performed twice a week for four weeks and a three to four days' interval between each session, with the 415 nm blue treatment head being used for the first treatment session followed by the 633 nm red treatment head for the second session each week.

### Instrumental Measurement

The moisture level, the sebum level, and the melanin level were measured in numerical values using a Corneometer™ (Courage+Khazaka, Koln, Germany), a Sebumeter™ (Courage+Khazaka), and a Mexameter™ (Courage+Khazaka), respectively. The measurements before treatment were carried out after a 15 minutes' stabilizing period to exclude any possible influences of outdoor activity on the skin condition, e.g. by sweating or flushing. The same part of the right malar area was chosen for the measurement every time to exclude any site-variation bias. The measurements were performed repeatedly at ten minutes after the end of treatment to exclude any possible effects of mild heat from the phototherapy device on the measured values.

### Statistical Analysis

The differences between before and after treatment in the moisture, sebum and melanin levels were analyzed using sign rank tests with the medians. Additionally, the differences in the melanin levels were also analyzed separately according to the wavelengths of light, namely blue and red light, using the same statistical method.

### Results and Discussion

Twenty four out of twenty seven subjects completed the whole study schedule. The data from the dropped-out subjects were excluded from the final analysis. The statistical analysis revealed that the melanin level had decreased significantly after the blue and red light combination LED phototherapy with a median of differences of −7.08 (p<0.005) (see Table 2). An additional statistical analysis was done to find out which wavelength of light had affected the melanin level more strongly (see Table 3). It revealed that the melanin level increased by 6.7 (the median of differences between before and after one treatment session) after blue light irradiation without a statistical significance (p-value>0.1), whereas it decreased by 15.5 with a statistical significance (p-value<0.005) after red light irradiation. The moisture and sebum levels were not significantly different between before and after treatment, though they showed a tendency to decrease slightly.

When asked to assess the subjects' global satisfaction levels, fourteen patients out of twenty four (58.3%) spontaneously reported that they had perceived brightening of their skin tone (complexion). This reported effect of brightening of the skin tone (complexion) may have some relationship with the reduction of the objectively-measured melanin levels.

TABLE 2
Statistical Analysis Results of the Differencecs
of Observed Melanin Levels Between Before and After
the Combined Blue and Red LED Phototherapy

| | | | p-value |
|---|---|---|---|
| Type of instrumental | Difference | | (Sign rank |
| measurement | Mean ± std | median | test) |
| Corneometer ™ (moisture) | −0.81 ± 4.34 | −1.42 | 0.3264 |
| Sebumeter ™ (sebum) | −13.88 ± 56.88 | −5.25 | 0.2502 |
| Mexameter ™ (melanin) | −5.69 ± 8.38 | −7.08 | 0.0032 |

TABLE 3
Statistical Analysis Results of the Differences in
the Melanin Level Between Before and After Each Blue
and Red Light Irradiation, and After the Final Treatment
Compared with Before the First Treatment

| | Difference | | |
|---|---|---|---|
| Variables | Mean ± std | median | P-value |
| Between before and after | 8.52 ± 15.48 | 6.70 | 0.3125^{†} |
| each blue light irradiation | | | |
| Between before and after | −17.97 ± 13.62 | −15.50 | 0.0020^{†} |
| each red light irradiation | | | |
| Between before the first | −17.79 ± 18.60 | −16.20 | 0.0001^{††} |
| treatment and after the final | | | |
| treatment | | | |

^{†}P-values are for sign rank test,
^{††}P-value is for paired t-test

### Example 2

### Skin Melanin Level Lowering Effects of Red Light LED Irradiation: Twice a Week

### Light Source

The phototherapy system used as the light source for this study consisted of a base and interchangeable heads emitting quasimonochromatic light of each different preset wavelength from adjustable planar arrays of LEDs. Red light and infrared light were used either alone or in combination according to each treatment protocol of the four arms of the study (see the ‘Study design’). The near infrared head (Omnilux Plus™, Photo Therapeutics Ltd., Fazeley, UK) comprised five articulated panels containing 108 LEDs each, so that they could be adjusted to fit the contour of the patient's face optimally. The red light head (Omnilux Revive™, Photo Therapeutics Ltd.) consisted of four panels containing 420 LEDs each arranged in the same way. The treatment heads delivered symmetrical peak wavelengths; 830±5 nm for the infrared light and 633±6 nm for the red light. The irradiance was 55 mW/cm² for the infrared light and 105 mW/cm² for the red light at a distance of 1 to 10 centimeters from the light source. The radiant fluences, or doses, during a single treatment for twenty minutes were 66 J/cm² and 126 J/cm² for the infrared and red treatment heads, respectively.

### Study Design

A total of 112 patients (2 males and 110 females), ranging in age from 35 to 55, with visible signs of aging were recruited for this study and randomly divided into four groups of 28 patients each. The number of subjects was calculated statistically (SAS version 9.1), so that this study would detect differences in the mean percentage improvements among the four different treatment groups when the maximum standardized effect size was larger than 0.5, at a 5% significance level, using an analysis of variance (ANOVA) with 80% power, allowing 10% extra for dropouts.

All patients were randomly divided using computer-generated random numbers into four groups of 28 patients each. Group 1 was treated with the 830 nm head alone, group 2 with the 633 nm head alone, group 3 with a combination of the 830 and 633 nm heads by alternating them in that order, and group 4 with a sham treatment light as the control group. The standby mode of the 633 nm LED head was used as the sham treatment. This study employed the split-face model; in all groups, the patients were treated only on the right half of the face with the left half being occluded.

The melanin level was measured by Mexameter™ (Courage+Khazaka, Koln, Germany) before treatment at every treatment session. After the measurement, each patient washed his/her face and was treated for twenty minutes in the supine position with the wavelength of light as set by the protocol of his/her group. The distance between the irradiating head and the patient's nose was about 3-5 cm. In group 3, we alternated sessions of the 830 nm and 633 nm LED treatment in succession, with the 830 nm treatment head being used first. Goggles were worn to protect the retinae from direct illumination. When the treatment was over, the instrumental measurements for the melanin level were performed in the same way as before treatment. In this manner, the therapy was performed twice a week for four weeks at a three to four-day interval between each session.

### Instrumental Measurements

The melanin level was measured by Mexameter™ (Courage+Khazaka, Koln, Germany) before treatment at every treatment session on both the exposed and the covered side of the facial skin of a given subject. The same area of each malar aspect was chosen for the measurements to avoid any site-variation bias.

### Tissue Assay

A total of 19 patients volunteered for punch biopsies; 5 in group 1, 6 in group 2, 5 in group 3, and 3 in group 4. 3 patients from group 1, 4 patients from group 2, 3 patients from group 3 and 1 patient from group 4 were selected, and 2 mm punch biopsies were performed on the lateral aspect of their right cheeks before the first treatment and 2 weeks after the last treatment. Schmorl's stain was performed to investigate any substantial change in the melanin amount in histological level.

### Statistical Analysis

The differences in the melanin levels between before and after each treatment were analyzed using sign rank tests with the medians.

### Results and Discussion

Only the data of group 2 showed a statistically significant decrease in the melanin levels after treatment compared to before treatment (see Table 4). The mean of the differences was −14.61±7.15 on the treated sides in group 2, while it was −1.84±7.59 in the covered sides in the same group. There was no statistically significant difference between before and after treatment both on exposed sides and covered sides in the other treatment groups or the control group, although a varied tendency was observed for the melanin levels to decrease slightly.

TABLE 4
Statistical Analysis Results of the Differences of
the Observed Melanin Levels Between Before and After
the Red Light Irradiation (Group 2) Compared with
Those of the Other Groups (Groups 1, 3 and 4)

| | | Differences | | |
|---|---|---|---|---|
| Group | | Mean ± std | Median | p-value^{†} |
| Group 1 | Covered | −5.43 ± 6.86 | −7.05 | 0.4663 |
| (830 nm alone) | Treated | −6.94 ± 7.66 | −8.95 | |
| Group 2 | Covered | −1.84 ± 7.59 | −1.89 | <0.0001* |
| (633 nm alone) | Treated | −14.61 ± 7.15 | −15.47 | |
| Group 3 | Covered | −6.27 ± 5.92 | −5.94 | 0.0955 |
| (830 nm and 633 | Treated | −9.74 ± 6.68 | −11.34 | |
| nm) | | | | |
| Group 4 | Covered | −3.38 ± 6.87 | −4.16 | 0.241 |
| (Control sham | Treated | −1.03 ± 5.24 | 0.84 | |
| light) | | | | |

^{†}P-values are for paired t-test
*Statistically significant

In the results of Schmorl's staining, it was observed that the amount of melanin pigment did not change significantly after the treatment. It is thought that the decrease in the melanin levels measured by Mexameter™ were possibly as the result of reasons other than the direct decrease in the amount of melanin pigment, for example, changes in the reflection of light from, the epidermis or alteration in the absorption and scattering characteristics of light in the dermis. Because the Mexameter™ quantifies melanin pigmentation by comparing the amount of reflected light to that of a device-generated reference beam, morphological changes in the epidermis and even in the dermis may alter this reflected beam and cause a decrease in the measured levels without any actual decrease in the total melanin amount. Because the human eyes also receive visual information by detecting the reflected light from the surface of a given object, any changes that alter the reflection of the light can affect the perceived appearance of the object. It is hypothesized that the reduction of the melanin levels measured by the Mexamter, or brightening of the skin tone (complexion) perceived by the subjects' eyes, might be caused by some optical alterations of the reflected light from the skin surface.

While this invention has been particularly shown and described with references to example embodiments thereof, it will be understood by those skilled in the art that various changes in form and details may be made therein without departing from the scope of the invention encompassed by the appended claims.

1. A method of reducing appearance of melanin on the skin of a subject, comprising exposing the skin to red narrow-band radiation at a wavelength(s) in a range of between 620 nm and 750 nm and having a band width of between 0 nm and 20 nm, in an effective dose to cause the appearance of the skin melanin to diminish and essentially not to cause photothermolysis of the skin.

2. The method of claim 1, wherein the red narrow-band radiation has a power density in a range of between 10 mW/cm² and 120 mW/cm².

3. The method of claim 2, wherein the power density is in a range of between 10 mW/cm² and 75 mW/cm².

4. (canceled)

5. The method of claim 1, wherein the skin exposure to the red narrow-band radiation per each dose lasts for less than 20 minutes.

6. The method of claim 5, wherein the skin exposure to the red narrow-band radiation per each dose lasts for between 5 minutes and 15 minutes.

7. The method of claim 1, further comprising adjusting exposure time and/or power density of the red narrow-band radiation according to skin phototype of the subject.

8. (canceled)

9. The method of claim 1, further comprising performing at least one treatment on the skin prior to the skin exposure to the red narrow-band radiation, the treatment being selected from the group consisting of physical microdermabrasion, exfoliating and chemical peel of the skin.

10. The method of claim 9, wherein the treatment is performed by at least one peeling means selected from the group consisting of a diamond peel, a crystal peel, a skin scrubber with an ultrasonic wave(s), a skin exfoliating means, a physical means of superficial skin resurfacing, skin rubbing with solid carbon dioxide, and a chemical peel.

11. The method of claim 10, wherein the treatment is a chemical peel performed with a composition comprising trichloroacetic acid, resorcinol, an alpha-hydroxy acid, a beta-hydroxy acid or a seaweed extract including an enzymatic exfoliating agent.

12.-13. (canceled)

14. The method of claim 1, further comprising applying a layer of a gel, cream or lotion on the skin prior to the skin exposure to the red narrow-band radiation, wherein the skin is exposed to the red-narrow band irradiation through the gel, cream or lotion layer.

15. The method of claim 14, wherein the gel, cream or lotion has at least 70% transparency at the red narrow-band radiation.

16.-18. (canceled)

19. The method of claim 1, wherein the red narrow-band radiation is non-coherent red narrow-band radiation.

20. The method of claim 19, wherein the non-coherent red narrow-band radiation is red light-emitting diode radiation.

21. A method of reducing appearance of melanin on the skin of a subject, comprising exposing the skin to non-coherent red narrow-band radiation at a wavelength(s) in a range of between 620 nm and 750 nm and having a band width of between 0.1 nm and 20 nm, in an effective dose to cause the appearance of the skin melanin to diminish.

22.-37. (canceled)

38. The method of claim 1, further comprising repeating exposure of the skin of the subject to the red narrow-band radiation at intervals of from 0.5 day to 10 days.

39. The method of claim 38, wherein the skin is exposed to the red narrow-band radiation one, two, three, four, five, six or seven times per week.

40. (canceled)

41. The method of claim 38, wherein the exposures, collectively referred to as “treatment period,” are repeated for a duration of between one week and twelve weeks.

42. (canceled)

43. The method of claim 1, wherein the band width of the red narrow-band radiation is between 0 nm and 15 nm.

44. The method of claim 43, wherein the band width of the red narrow-band radiation is between 0.1 nm and 10 nm.

45. (canceled)

46. The method of claim 1, wherein the wavelength(s) is in a range of between 625 nm and 650 nm.

47. (canceled)

48. The method of claim 46, wherein the red narrow-band radiation is at 633 nm.

49. The method of claim 1, wherein the skin is exposed to the red narrow-band radiation with the energy density of between 10 J/cm² and 200 J/cm² per single dose.

50. The method of claim 49, wherein the skin is exposed to the red narrow-band radiation with the energy density of between 10 J/cm² and 100 J/cm² per single dose.

51. (canceled)

52. The method of claim 1, further comprising applying a layer of a cosmetic composition prior to, or after, the skin exposure to the red narrow band radiation, the cosmetic composition comprising at least one skin-brightening agent selected from the group consisting of an alpha-hydroxy acid, a beta-hydroxy acid, hydroquinone, kojic acid, azelaic acid, licorice P-T, arbutin, melawhite, ascorbic acid, vitamin C, magnesium-L-ascorbyl-2-phosphate and corticosteroid.

53. A kit, comprising:
a) a radiation source generating red narrow-band radiation at a wavelength(s) in a range of between 620 nm and 750 nm, the narrow band radiation having a band width less than 20 nm and having a power density in a range of between 10 mW/cm² and 120 mW/cm²; and
b) a manual instructing a user how to use the red narrow-band radiation for red narrow-band irradiation treatment to reduce appearance of melanin on the skin of a subject.

54.-72. (canceled)
